(19) United States
(12) Patent Application Publication
(10) Pub. No.: US 2008/0187574 A1
(43) Pub. Date: Aug. 7, 2008

(54) Mycellated grain and other myceliated agricultural materials to be used as animal food supplement

(76) Inventor: John C. Holliday, Carson City, NV (US)

Correspondence Address: GABOR L. SZEKERES, P.O. BOX 27938, ANAHEIM HILLS, CA, 92809, US

(21) Appl. No.: 12/012,358

(22) Filed: Feb. 1, 2008

Related U.S. Application Data
(60) Provisional application No. 60/899,514, filed on Feb. 5, 2007.

(51) Int. Cl. A23K 1/17; A61K 36/06; C12N 1/00

(52) U.S. Cl. 424/442; 424/195.15; 435/256.8

(57) ABSTRACT

Animal feedstuff having beneficial properties is obtained by adding to a substrate one or more fungal species of the kind that excretes substances into said substrate during its growth which are beneficial to the health, growth or weight gain of an animal, or animals to which the feedstuff is intended and allowing the fungus to grow and/or ferment on the substrate. Suitable substrates are cereal grains, residue of cereal grains, agricultural primary products, agricultural waste products, and other cellulosic materials or a combination of one or more of the same. Suitable fungus species include Cordyceps species, Ganoderma species, Grifola species, Trametes Species, Lentinula species, Antrodia species, Agaricus species, Tremella species, Pleurotus species, Lentinus species, Polypore species, Agaricales species, Ascomycetes species and Basidiomycetes species. Some substrates such as certain agricultural waste products and cellulosic material are not suitable for animal consumption per se but become suitable as a result the fungal growth and fermantation by the fungus or fungi on and in them.

### CLAIM OF PRIORITY

The present application claims the priority of U.S. provisional application Ser. No. 60/899,514, filed on Feb. 5, 2007.

### BACKGROUND OF THE INVENTION

1. Field of the Invention

The present invention is directed to myceliated grain or other myceliated agricultural material to be used as animal feed supplement. More particularly, the present invention is directed to a substrate which is composed of grain, grain residue or other agricultural materials fermented by various species of fungi, and containing or admixed with fungal biomass to be used as supplement for animal feed to improve the health and/or cause increased feed conversion and weight gain or other physiological changes in the animals that consume the supplement.

2. Brief Description of Prior Art

The prior art has utilized extracts of fungi mass as an animal feed supplement per as a substitute for antibiotics. Such use is described or mentioned in a publication by Willis, Isikhuemhen and Ibrahim, titled “Probiotics and Mushroom Extracts Alone or in Combination with Probiotics”, Poultry Science 2007. 86:1856-1860 and in a publication by Jong Ho Koh et al. titled “Hot-water extract from mycella of Cordyceps sinensis as a substitute for antibiotoc growth promoters” Biotechnology Letters 25: 585-590, 2003. However, as far as the present inventor is aware, there has not been any use as animal feed supplement of digestible or undigestible material (such as starchy or cellulosic material) on which a fungal species had been deliberately grown. On the contrary, it was and is common practice in the state of the art to either discard or purify from fungal mass (such as mold) animal feedstuff.

The following general scientific knowledge comprises further background to the invention, although it is asserted that recognizing the applicability of this knowledge for the purposes described in this application is not prior art and presents only the theoretical basis of the present invention.

In the kingdom of fungi, the majority of secondary metabolites produced by the saprophytic fungi are bioactive compounds which are extra-cellular in nature, that is, they are excreted out of the cells of the fungus into the surrounding environment. This is due to the unique nature of saprophytic fungi with their external digestive mechanism. Examples of this modus vivendi of fungi can be seen in the way fungi digest their food source, first by excreting digestive compounds that digest the food source in situ, and excreting other compounds which act as transport molecules bringing the nutrients back across the cell walls of the fungal cells. Fungi also excrete compounds to stun or kill bacteria and other organisms to give the fungi an advantage in the food source. The latter compounds are termed “antibiotics” with penicillin serving as the classic example. Other compounds excreted by fungi can be antifungal (against another species) antiviral, antihelminetic, immune enhancing or have other biological or physiological effects. Thus, the bioactivities of fungi takes place in large measure outside the fungal cell boundary and affect the surrounding environment in subtle or significant ways.

### SUMMARY OF THE INVENTION

In accordance with the present invention the advantageous properties of certain saprophytic fungi species can be used in the artificial production of animal feed which itself may compose, without limitation, cereal grains such as millet, milo (sorghum), rice, maize, wheat, rye, rye grass seed, oats or other cereal grains, The residue from cereal grains such as the hull or husk, the bran, the stems and roots, as well as other agricultural primary products or agricultural waste products. The animal feed stuff, which may be used in accordance with the present invention in combination with fungal biomass can also be based upon agricultural products or waste by-products such as, but not limited to brewer's grain, coffee processing waste, maize stalks, banana leaves, cereal grain straw, grass or grass hay, sawdust or other cellulosic or ligno-cellulosic material, and cereal based industrial processing wastes. In addition to the feedstuffs listed above, combinations of cereal grain and agricultural waste can be used together, either as the substrate upon which the fungal biomass is grown, and/or in combination with externally grown fungal biomass. Also paper or cardboard or sawdust or other cellulosic materials can be used as feed substrates when properly prepared through fungal fermentation according to this invention, alone or in combination with the fungal biomass. By utilizing this invention nearly any agriculture product or byproduct can be used as the substrate in and upon which the fungal biomass is grown when said agricultural byproduct is properly prepared. Thus, the agricultural product or byproduct, which may or may not be suitable for use as an animal feed in its own original form, is biologically converted in accordance with the present invention by fungi into a usable feedstuff material containing proteins, carbohydrates and lipids for caloric and nutrient value that also has the additional advantage of having certain advantageous biological, biochemical and physiological properties, such as providing immune enhancement for resistance to disease, increased feed conversion rates, antibiotic and antimicrobial substitute properties or other biological activity. In short, the invention is a solid state fermentation process, whereby grain or other agricultural materials is converted into a feedstuff that has considerably increased value to the animal than the original, unfermented, unconverted substrate had of its own merit. The present invention provides means for converting indigestible agricultural material which is unsuitable as animal feed into suitable feedstuff, and increases the biological activity of the feedstuff, whether indigestible or digestible in its original state, through the fungal bioconversion and accumulation of fungal secondary metabolites.

### DETAILED DESCRIPTION OF THE INVENTION

In light of what is stated in the foregoing Background and Summary of the Invention, when one or more of the fungi species described below are allowed to grow on and in one or more of the above-mentioned substrates, the substrate or feedstuff itself is partially digested by the fungal species or species, resulting in degradation of the original properties of the substrate, and bioconversion of that material into fungal tissue, either wholly or in part, which substrate or feedstuff material then has different properties than the original substrate or feedstuff material. Further, the converted substrate or feedstuff material upon and into which the fungal culture is grown absorbs the above-noted extra-cellular secondary metabolic substances having beneficial biological or physiological activities, that have been excreted by the fungal cells in the process of digesting the substrate. These secondary fungal metabolites (substances) combined with the nutritional and caloric content represented by the converted and unconverted portion of the substrate result in an animal feedstuff with properties superior to the original substrate. This is because in the product of the invention obtained as a result of fungal growth the beneficial properties of the fungal extra-cellular metabolites, such as the antibiotic, antiviral, antifungal, and immune enhancing properties co-exist with the nutritional and caloric content of the original feedstuffs and with the fungal tissue. Using this product of the invention as feedstuff provides a synergistic benefit to the animal, in that the use of externally supplied antibiotics, antivirals and other medications can be partially or totally eliminated, those functions being performed by the secondary fungal metabolites present in the feedstuffs of the invention.

The actual substrate (original feedstuff) is chosen based on the considerations of the type of fungus to be used to grow on and in the substrate and the type of animal or animals for which the feedstuff of the invention (hereinafter myceliated feedstuff) is intended. Suitable substrates have been described above.

The chosen substrate can be advantageously, but not necessarily, mixed with various substances to alter its overall physical or chemical characteristics. For example, calcium carbonate or other substance can be admixed with the substrate to act as a pH buffer to provide more optimal conditions for growth of the target fungus. Another example would be gypsum that can be admixed to cause a modification of the physical structure of the substrate for the same reason. Various minerals, carbohydrates, proteinaceous or other substances can be added to improve the nutritional value of the substrate and/or to optimize growth of the target fungus.

The chosen substrate is usually mixed with a pre-determined amount of water and may, but not necessarily, be cooked to establish the correct water content and physical and chemical properties for optimal growth of the chosen fungus species. The cooked or uncooked substrate then is sterilized using heat, chemical, UV, radiation or other known means, resulting in a pH balanced, nutritionally complete, microbe-free, nutritionally modified sterile substrate upon which one or more specially chosen targeted fungal strains are grown through the mechanism of solid state fermentation in accordance with the invention.

The chosen fungal strains of the phyla Ascomycota or Basidiomycota are then inoculated under sterile condition into or onto the substrate prepared as described above, and the target fungus is allowed to grow for a period of time under controlled climatic and environmental conditions chosen for optimum growth according to the fungal species involved, for a period of time determined by the fugal species and substrate chosen, resulting in conversion of some or all of the substrate into fungal tissue and the accumulation in the mass of extra-cellular compounds (fungal secondary metabolites) of particular interest from a bioactivity perspective. Typically the fungus or fungi are allowed to grow and/or ferment on the substrate for 1 to 100 days. More likely the time range is 5 to 60 days, and still more likely the time range is 28 to 31 days.

After the substrate is converted sufficiently according to the target species of animal to be fed, the fungal biomass, with or without remaining unconverted substrate is harvested. Preferably the fungal biomass is harvested and used together with the remaining unconverted substrate. The harvested mass is processed by either drying, or by drying and grinding, or frozen, or refrigerated or fed fresh to the animals. This processed or fresh material comprising fungal biomass, fungal secondary metabolites and remaining unconverted substrate is fed to animals with the intent of providing some or all of the animal nutritional requirements and health and growth benefits. One of the health benefits can be a host mediated immune response, which is thought to be triggered partially, but not necessarily in entirety, by the fungal-generated polysaccharides and extra-cellular compounds.

The species of fungi that can be used in accordance with invention are as follows:

Cordyceps sinensis and other Cordyceps species;
Ganoderma lucidum and other Ganoderma species;
Grifola frondosa and other Grifola species;
Trametes versicolor and other Trametes Species;
Lentinula edodes and other Lentinula species;
Antrodia camphorate [also known as Antrodia cinnamomium] and other Antrodia species;
Agaricus braziliensis and other Agaricus species;
Tremella mesentarica or other Tremella species;
Pleurotus ostreatus and other Pleurotus species;
Lentinus squarrosulus and other Lentinus species;
Various other Polypore species;
Various other Agaricales species;
Various other Ascomycetes species;
Various other Basidiomycetes species:

Examples of animal species that can be fed with the myceliated grain feed supplement of the invention and are likely to benefit from it are: dogs, cats, other companion (pet) animals, cows, horses, pigs, sheep, goats, rabbits, chickens and other fowl, farmed fish, zoo animals and other animals intended for human food. In accordance with the invention, because of its fungal biomass the myceliated feedstuff serves not only as a caloric source but also as a medicinal supplement to promote health, in some cases stimulate growth and weight gain and is likely to reduce or eliminate the need for externally supplied antibiotics in most animals under most conditions, since the feedstuff of this invention contains varied and multiple naturally occurring antibiotic and antimicrobial substances due to the particular process of growing said fungal species directly in and on the substrate with the resultant accumulation of those secondary fungal metabolites of interest.

In accordance with the invention the dosage of myceliated grain or myceliated feedstuff that is to be fed to the targeted animal(s) should be such that the fungal biomass itself (not the total myceliated grain or myceliated feedstuff) should represent at least 1% of total food intake of the animal as measured by caloric content. Preferably, it should represent approximately 1-5% of total food intake of the animal as measured by caloric content, and at a maximum it should represent 5-100% of the total food intake of the animal. Thus, in accordance with the present invention the fungal biomass may represent approximately 1 to 100% of the food intake of the animal, as measured by caloric content. Those skilled in the art will readily understand that selection of the dosage depends upon the species of animal, species of fungus or fungi and original substrate as well as the degree of substrate conversion caused by fungal growth.

In the course of developing this invention, many animals were tested to insure that the myceliated supplement does in fact provide the health benefits claimed herein. Below is a description of the processes of making the myceliated feedstuff and of some of the trials conducted in testing and verifying their utility and advantages when fed to animals.

Trial #1—Colorado Natural Beef Field Trial—

In this trial, the results of which are summarized in Table 1, the number of calves tested was 599 total, 180 in control group and 419 in test group. All animals were vaccinated and wormed, no antibiotics or growth hormones were used in any animal. The test period was 30 days. All calves were 10 days old+/−3 days. Dose given of myceliated grain supplement was 1 oz/head/day×4 days. Total dose 4 oz/head. More specifically, the animals were fed the myceliated feedstuff supplement for four days in the doses stated above. However, the study was conducted for 30 days in that the results, such as weight gain and resistance to disease and other changes in the animals (as applicable) were observed for the above-noted 30 days after the beginning of feeding the myceliated feedstuff as a supplement. This explanation regarding dosage and length of the study applies to each of the trials described in this application for patent, unless stated or from the context appears otherwise. The myceliated feedstuff supplement was added as top dressing to normal feed. The myceliated feedstuff (myceliated grain supplement) was grown on sorghum and other grain, such as one of rye, millet, oats, corn and wheat or combination of two or more of these grains, with the fungal species used for fermentation being Lentinus edodoes, Cordyceps sinensis, Agaricus subrufecense, Trametes versicolor, Grifola frondosa, Ganoderma lucidum in equal proportions, grown for 30 days prior to drying and grinding and using as a feed supplement for this study.

TABLE 1

| | Number of | | | Weight Gain |
|---|---|---|---|---|
| Group | animals | Illness | Death | in 30 days |
| Control | 180 | 15.5% | 5.5% | 1.85 lbs/day |
| Test | 419 | 3.1% | 0 | 3.05 lbs/day |

As is shown in Table 1 there was marked improvement in both health and weight gain parameters in the myceliated feedstuff supplemented group when compared to the control group.

Trial #2—High Stress Calf Barn Study—

This study, the results of which are summarized in Table 2, was run on calves considered salvage due to bovine viral respiratory disease, that is calves that were not considered healthy enough to be grown into beef cattle, but were rather slated for use in dog food or other salvage meat processing. Total number of calves in study was 75 with the control group comprising 26 and the test group composed of 49 calves. Supplement was added as top dressing to normal feed. Myceliated grain supplement was grown on sorghum and other grain, the examples without limitation of which are specified above in Trial 1, with the fungal species used for fermentation being Lentinus edodoes, Cordyceps sinensis, Agaricus subrufecense, Trametes versicolor, Grifola frondosa, Ganoderma lucidum in equal proportions, grown for 30 days prior to drying and grinding and using as a feed supplement for this study. The calves in this study were given myceliated grain 1 oz/head×3 days, followed by 1 oz./head on revaccination day. Total 4 oz/head.

89% of the calves in the control were treated for Bovine Viral Respiratory Disease (BVRD). All calves in this group suffered eye infections. In the test group 12% of the animals suffered eye infections only, all of which cleared up upon treatment. There was no incidence of BVRD.

All calves in the control group and in the test group were preconditioned with Micotil®, vaccinations and worming. All calves were light weight, averaging 450-500 lbs. and 90% (all male calves) required castration and worming.

TABLE 2

| | | | | 30 day |
|---|---|---|---|---|
| | Number | | | Weight |
| Group | of animals | Illness | Death | Gain |
| Control | 26 | 89% | 19% | 1.0 lbs/ |
| | | | | day |
| Test | 49 | 12%* | 0 | 2.3 lbs/ |
| | | | | day |

As is shown in Table 2 the calves in the test group receiving the myceliated feedstuff of the invention showed 84% decreased morbidity and 100% decreased mortality. Initial 30 day weight gains are 80% above historical averages for this calf salvage operation.

Trial #3—Ohio Salebarn Calf Study—

In this trial, the results of which are summarized in Table 3, we tested 614 calves total, 585 in the test group and 29 in the control group. These animals were all given vaccinations, antibiotic (penicillin) and wormer (Ivomec). All deaths were due to BRVD. Trial calves were given myceliated feedstuff (myeceliated grain) 1 oz/head/day×3 days, followed by 1 oz./head on day 12 with revaccination. Total dose 4 oz/head. Supplement was added as top dressing to normal feed. Myceliated grain supplement was grown on sorghum and other grain, the examples without limitation of which are specified above in Trial 1, with the fungal species used for fermentation being Lentinus edodoes, Cordyceps sinensis, Agaricus subrufecense, Trametes versicolor, Grifola frondosa, Ganoderma lucidum in equal proportions, grown for 30 days prior to drying and grinding and using as a feed supplement for this study.

TABLE 3

| | Number of | | | 30 day |
|---|---|---|---|---|
| Group | animals | Illness | Death | Weight Gain |
| Control | 29 | 83% | 24.1% | 0.9 lbs/day |
| Test | 585 | 2.6% | 0 | 3.1 lbs/day |

As is shown in Table 3 illness and death of the calves in the study group were dramatically reduced and their weight gain increased relative to the control group.

Trial #4—Field Trial of Side-by-Side Dairies in Central California as Cattle Antibiotic Replacement (CAR)—

This study, the results of which are shown in Table 4, compares 361 dairy calves on two dairies located within five miles of each other, which share many commonalities. These include common gene pool, common methodology and handling of calves and each use the same calf raiser. The historical averages for the previous nine years were available for the two dairies and each dairy showed similar rates of illness and mortality. For this study all the calves from one dairy were given the trial protocol and the other dairy was used as the control. The dosage used for this trial was 1 oz/head/day×2 days, followed by 0.5 oz/head×1 day. Total 2.5 oz/head. The myceliated feedstuff (myceliated grain) supplement was added as top dressing to normal feed. The myceliated grain supplement was grown on sorghum and other grain, the examples without limitation of which are specified above in Trial 1, with the fungal species used for fermentation being Lentinus edodoes, Cordyceps sinensis, Agaricus subrufecense, Trametes versicolor, Grifola frondosa, Ganoderma lucidum in equal proportions, grown for 30 days prior to drying and grinding and using as a feed supplement for this study

As seen from the data, the results were significant.

TABLE 4

| | Number | | Death | | Reduction |
|---|---|---|---|---|---|
| | of | Death Rate | during | Weight | in |
| Group | animals | (Historical) | study | Gain | Mortality |
| Dairy A | 180 | 11.5% | 16.7% | Not | N/A |
| Control | | | | Given | |
| Dairy B | 181 | 10.8% | 1.7% | Not | Reduced |
| Test | | | | Given | by 85% |

This trial continued for about 34 months. It is to be noted as proof of efficacy of the product of the invention that when the test dairy ran out of product and was not using it for about two months (the manager went on vacation and forgot to reorder the supplement) the mortality rate jumped from 1.7% to 15.65% (18 calves died out of 118 born during this period) After an approximately two months hiatus the test diary re-started treating the calves with the myceliated feedstuff supplement again the mortality has dropped to 2.86%.

Trial # 5—Nebraska Feedlot Study—

In this trial, the results of which are summarized in Table 5, we tested myceliated feedstuff (myceliated grain) supplement on 1099 calves and compared the results to the historical average for this feedlot for the previous nine years. Trial calves were given myceliated grain 1 oz/head/day×3 days, followed by 1 oz./head on day 12. Total dose 4 oz/head. The myceliated feedstuff supplement was added as top dressing to normal feed. Myceliated grain supplement was grown on sorghum and other grain, the examples without limitation of which are specified above in Trial 1, with the fungal species used for fermentation being Lentinus edodoes, Cordyceps sinensis, Agaricus subrufecense, Trametes versicolor, Grifola frondosa, Ganoderma lucidum in equal proportions, grown for 30 days prior to drying and grinding and using as a feed supplement for this study.

TABLE 5

| | Number of | | |
|---|---|---|---|
| Group | animals | Illness | Death |
| Control | 9000/yr × 9 yrs | 13% | 1.5% |
| | (historical | | |
| | data) | | |
| Test | 1099 | 0.003% | 0 |

As Table 5 demonstrates the number of illnesses and death in the test group that has received the myceliated feedstuff of the invention was drastically reduced when compared to the historical control group.

Trial # 6—Milk Quality Trial, Tulare California—

In this study, the results of which are summarized in Table 6, we tested the effect of adding myceliated feedstuff (myceliated grain) to cattle feed to increase milk quality as determined by reduction in Somatic Cell Count, with lowered somatic cell counts indicating lowered mastitis and increased milk quality. 26 lactating dairy cows were used in this trial, 13 as control group receiving no supplement and 13 given 1 oz of myceliated grain per day during the course of this four week study. The myceliated grain supplement was added as top dressing to normal feed. The Myceliated grain supplement was grown on sorghum and other grain, the examples without limitation of which are specified above in Trial 1, with the fungal species used for fermentation being Lentinus edodoes, Cordyceps sinensis, Agaricus subrufecense, Trametes versicolor, Grifola frondosa, Ganoderma lucidum in equal proportions, grown for 30 days prior to drying and grinding and using as a feed supplement for this study.

TABLE 6

| | | | 4 weeks | |
|---|---|---|---|---|
| | | Initial | later | |
| | | Somatic | Somatic | |
| | Number | Cell | Cell | |
| | of | Count - | Count - | Percent |
| Group | animals | Avg. | Avg | Change |
| Control | 13 | 1,854,811 | 2,374,000 | +22% |
| Test | 13 | 2,049,636 | 957,455 | −54% |

As Table 6 demonstrates the number of somatic cells decreases significantly in the milk of the cows which have been fed the myceliated feedstuff supplement of the invention.

Trial # 7—Natural Beef Study Montana—

In this study, summarized in Table 7, 498 cattle were tested, with 318 receiving 1 oz of myceliated grain daily, while 180 head of cattle were used as the control group. The myceliated feedstuff supplement (myceliated grain) was added as top dressing to normal feed. The myceliated grain supplement was grown on sorghum and other grain, the examples without limitation of which are specified above in Trial 1, with the fungal species used for fermentation being Lentinus edodoes, Cordyceps sinensis, Agaricus subrufecense, Trametes versicolor, Grifola frondosa, Ganoderma lucidum in equal proportions, grown for 30 days prior to drying and grinding and using as a feed supplement for this study. The trial lasted 35 days for the control group and 30 days for the study group.

TABLE 7

| | | | Average | | | | |
|---|---|---|---|---|---|---|---|
| | | | weight | | | | |
| | | | at the | | | | |
| | | | end of | | | | |
| | | | study | | | | |
| | | | (35 days | | | | |
| | | | for | | Average | | |
| | | | Control | | net gain | | |
| | | Average | Group, | | (rounded | | |
| | | Weight at | 30 days | Average | to whole | Percentage | Percentage |
| | Number | the | for | daily | lb) | Mortality | Morbidity |
| | of | beginning | study | weight | during | during | during |
| Group | animals | of study | group) | gain | the trial | trial | trial |
| Control | 180 | 535 lbs | 600 lbs | 1.85 lbs | 65 lbs | 5.5% | 15.5% |
| Study | 318 | 565.5 lbs | 657 lbs | 3.05 lbs | 91 lbs | 0% | 3.7% |

As the data in Table 7 demonstrate, the cattle in the study group receiving the myceliated feedstuff of the invention suffered lowered morbidity and mortality and had a significantly higher weight gain than the cattle in the control group.

Trial # 8 Calf Blood Chemistry and Weight Gain Study—

In this trial, summarized in Table 8, we tested 12 calves, 6 in the control group and 6 in the test group. Blood was drawn twice daily, morning and evening, and analyzed for cortisol and insulin content. The myceliated feedstuff supplement (myceliated grain) was added as top dressing to normal feed. The myceliated grain supplement was grown on sorghum and other grain, the examples without limitation of which are specified above in Trial 1, with the fungal species used for fermentation being Lentinus edodoes, Cordyceps sinensis, Agaricus subrufecense, Trametes versicolor, Grifola frondosa, Ganoderma lucidum in equal proportions, grown for 30 days prior to drying and grinding and using as a feed supplement for this study.

Cortisol is a stress hormone known to reduce immune function as cortisol levels elevate. Lowered cortisol levels tend to indicate increased immune function. Also measured were insulin levels and weight gain. This trial lasted 12 days and weight gain monitored for 60 days. We found that the application of 1 oz per day of myceliated grain to the calf's food resulted in lowered cortisol levels and increased insulin levels, which resulted in greater weight gain in the treatment group then the control group. Cortisol is measured in mcs/dl and insulin is measured in microIU/ml and the numbers in Table 8 refer to these units, as applicable.

TABLE 8

| | | | Average | Average |
|---|---|---|---|---|
| | | Average | insulin | weight |
| | | cortisol | level at | gain per |
| | Number | level at | the end | day |
| | of | the end of | of the | during |
| Group | animals | the trial | trial | the trial |
| Control | 6 | 34.337 | 18.254 | 1.2 lbs |
| Study | 6 | 21.489 | 29.392 | 2.2 lbs |

Trial #9—Rabbit Breeding Study—

In this trial, summarized in Table 9, 750 breeder rabbits were tested in three groups: One control group of 250 rabbits was given standard rabbit feed and another 250 rabbits were given the same feed except that 5% of the wheat middlins (roughage filler used in manufacturing the rabbit feed) was replaced with 5% myceliated grain. The myceliated grain supplement for this trial was incorporated into the rabbit feed pellets at a commercial mill, and consisted of six species of fungi grown on sorghum and other grain with fungal species Lentinus edodoes, Cordyceps sinensis, Agaricus subrufecense, Trametes versicolor, Grifola frondosa, Ganoderma lucidum in equal proportions, grown for 30 days prior to drying and grinding and using as a feed supplement for this study. A third group of 250 rabbits was also tested simultaneously with a different myceliated grain supplement comprised of 10% of the wheat middlins being replaced by 10% of Cordyceps sinensis converted sorghum grain, grown for 42 days before being dried and ground. This myceliated supplement was incorporated into the rabbit feed pellets at a commercial feed mill. The trial lasted 5 months. This trial was to determine the effect of myceliated grain on the breeding potential of commercially raised meat rabbits.

TABLE 9

| | | Average |
|---|---|---|
| | Number | live birth |
| | of | # per |
| Group | animals | litter |
| Control | 250 | 7.9 |
| Trial | 250 | 8.5 |
| Group | | |
| # 1 | | |
| Trial | 250 | 8.6 |
| Group | | |
| # 2 | | |

The two trial groups had higher number of young born than the control group. In the control group the average live birth number per litter was 7.9 and in the trial group #1 the average live birth number was 8.5 In the trial group #2 the live birth number per litter was 8.6. There were no other statistically significant differences seen in the two groups as far as survivability or weight gain differences.

What is claimed is:

1. An animal feed supplement comprising:
a substrate either directly edible by the target animal for which the supplement is intended or made edible by the animal for which the supplement is intended through a process of fungal solid state fermentation, and having nutritional and caloric value for the animal, and
a fungal mass, said fungus having been grown directly on and in the substrate, and wherein the fungus is of a species that excretes substances into said substrate during its growth which are beneficial to the health, growth or weight gain of the animal, said beneficial substances being included in the feed supplement.

2. An animal feed supplement in accordance with claim 1 where the fungus is one or more species selected from group consisting of Cordyceps species, Ganoderma species, Grifola species, Trametes Species, Lentinula species, Antrodia species, Agaricus species, Tremella species, Pleurotus species, Lentinus species, Polypore species, Agaricales species, Ascomycetes species and Basidiomycetes species.

3. An animal feed supplement in accordance with claim 2 where the fungus is one or more species selected from group consisting of Lentinus edodoes, Cordyceps sinensis, Agaricus subrufecense, Trametes versicolor, Grifola frondosa and Ganoderma lucidum.

4. An animal feed supplement in accordance with claim 3 where the supplement comprises all of the six the fungal species of Lentinus edodoes, Cordyceps sinensis, Agaricus subrufecense, Trametes versicolor, Grifola frondosa and Ganoderma lucidum.

5. An animal feed supplement in accordance with claim 4 where the supplement comprises all of the six the fungal species of Lentinus edodoes, Cordyceps sinensis, Agaricus subrufecense, Trametes versicolor, Grifola frondosa and Ganoderma lucidum in substantially equal amounts.

6. An animal feed supplement in accordance with claim 1 where the substrate is one or more substances selected from the group consisting of cereal grains, residue of cereal grains, agricultural primary products, agricultural waste products, and other cellulosic materials.

7. An animal feed supplement in accordance with claim 1 where the substrate comprises cereal grain.

8. An animal feed supplement in accordance with claim 1 where the substrate comprises agricultural waste product.

9. An animal feed supplement in accordance with claim 1 where the substrate comprises one or more cellulosic material selected from the group consisting of paper, cardboard and sawdust.

10. An animal feed supplement in accordance with claim 6 where the fungus is one or more species selected from group consisting of Cordyceps species, Ganoderma species, Grifola species, Trametes Species, Lentinula species, Antrodia species, Agaricus species, Tremella species, Pleurotus species, Lentinus species, Polypore species, Agaricales species, Ascomycetes species and Basidiomycetes species.

11. An animal feed supplement in accordance with claim 10 where the supplement comprises all of the six the fungal species of Lentinus edodoes, Cordyceps sinensis, Agaricus subrufecense, Trametes versicolor, Grifola frondosa and Ganoderma lucidum.

12. An animal feed supplement in accordance with claim 11 where the supplement comprises all of the six the fungal species of Lentinus edodoes, Cordyceps sinensis, Agaricus subrufecense, Trametes versicolor, Grifola frondosa and Ganoderma lucidum in substantially equal amounts.

13. An animal feed supplement comprising:
a substrate comprising one or more substances selected from the group consisting of cereal grains, residue of cereal grains, agricultural primary products, agricultural waste products, and other cellulosic materials, and
a fungal mass, said fungus having been grown directly on and in the substrate, and wherein the fungus is of a species that excretes substances into said substrate during its growth which are beneficial to the health, growth or weight gain of the animal, said beneficial substances being included in the feed supplement, said fungus being one or more species selected from group consisting of Cordyceps species, Ganoderma species, Grifola species, Trametes Species, Lentinula species, Antrodia species, Agaricus species, Tremella species, Pleurotus species, Lentinus species, Polypore species, Agaricales species, Ascomycetes species and Basidiomycetes species.

14. An animal feed supplement in accordance with claim 13 where the fungus is one or more species selected from group consisting of Lentinus edodoes, Cordyceps sinensis, Agaricus subrufecense, Trametes versicolor, Grifola frondosa and Ganoderma lucidum.

15. An animal feed supplement in accordance with claim 14 where the supplement comprises all of the six the fungal species of Lentinus edodoes, Cordyceps sinensis, Agaricus subrufecense, Trametes versicolor, Grifola frondosa and Ganoderma lucidum.

16. An animal feed supplement in accordance with claim 14 where the supplement comprises all of the six the fungal species of Lentinus edodoes, Cordyceps sinensis, Agaricus subrufecense, Trametes versicolor, Grifola frondosa and Ganoderma lucidum in substantially equal amounts.

17. A method of converting a substrate comprising one or more substances selected from the group consisting of cereal grains, residue of cereal grains, agricultural primary products, agricultural waste products, and other cellulosic materials into animal feedstuff, the method comprising the steps of
adding one or more species of fungus to the substrate, the fungus being one or more of a species that excretes substances into said substrate during its growth which are beneficial to the health, growth or weight gain of an animal to which the feedstuff is to be fed;
allowing the fungus to grow on the substrate and thereby allowing the fungus to excrete the beneficial substances to the substrate.

18. A method in accordance with claim 17 wherein the substrate is one or more species of grain.

19. A method in accordance with claim 17 wherein the substrate is one or more substances selected from agricultural waste product and other cellulosic material and wherein the process of allowing the fungus to grow converts said substrate into feedstuff suitable for feeding an animal.

20. A method in accordance with claim 17 where the fungus is one or more species selected from group consisting of Cordyceps species, Ganoderma species, Grifola species, Trametes Species, Lentinula species, Antrodia species, Agaricus species, Tremella species, Pleurotus species, Lentinus species, Polypore species, Agaricales species, Ascomycetes species and Basidiomycetes species.

21. A method of providing a nutritional and health benefit to an animal by feeding to said animal a feed supplement defined by claim 1.
